(19) United States
(12) Patent Application Publication
(10) Pub. No.: US 2019/0345531 A1
(43) Pub. Date: Nov. 14, 2019

(54) SPECTROMETER COMPATIBLE VACUUM AMPOULE DETECTION SYSTEM FOR RAPIDLY DIAGNOSING AND QUANTIFYING VIABLE BACTERIA IN LIQUID SAMPLES

(71) Applicant: AMT Scientific LLC, Halethorpe, MD (US)

(72) Inventors: He Zheng, Halethorpe, MD (US); Shengjun Zhang, Halethorpe, MD (US)

(21) Appl. No.: 15/973,619

(22) Filed: May 8, 2018

(51) Int. Cl. C12Q 1/06; G01N 21/59; G01N 33/18; B01L 3/00

(52) U.S. Cl. CPC C12Q 1/06; G01N 21/59; B01L 2300/0835; B01L 3/508; G01N 33/18

(57) ABSTRACT

Vacuum ampoule detection system detects and quantifies viable bacteria in liquid samples. Vacuum ampoules that include a supporting medium, a selective reagent, and a detecting reagent are useful in the rapid detection and quantification of viable heterotrophic bacteria in liquid samples. Vacuum ampoule detection system is suitable for the detection of total bacteria, E. coli, total coliform, etc. The vacuum ampoule detection system is also compatible with common spectrometer for visible light, UV light and fluorescence which can give more accurate analysis of the concentration of bacteria in the liquid sample.

### FIELD OF THE INVENTION

This invention relates to the field of rapid diagnosis of bacteria. More specifically, the invention comprises a self-filling vacuum ampoule system which optionally replaces the conventional techniques.

### BACKGROUND OF THE INVENTION

Bacterial contamination of drinking water supplies can cause gastrointestinal disease, impairments of cells of the digestive tract and organs, and life-threatening infections in people with depressed immune systems (EPA, 2011). In the case of human health, infection by gram-negative bacteria, such as Escherichia coli (E. coli) can cause urinary tract infection (UTI). Alternative approaches detecting total bacteria in liquid samples have been developed in the past few years. The dip-slide method has been approved by Environmental Protection Agency (EPA) (Federal Register 40 CFR Parts 141 and 143) that can give a semi-quantitative estimation of total bacteria in sample in 24-48 hours. However, because the volume of liquid analyzed is unrepresentative and not repeatable (˜1 mL), the accuracy and consistency of this method are fairly low. ATPmetry is an easy-to-operate method and can give results very quickly. However, it has the same issue as the dip-slide method that the sample volume is very small (˜100 μL) that is not representative. In addition, the reagents used in ATPmetry method require low temperature conservation that is not convenient for the field test. EPA has covered several PCA-based techniques to detect total bacteria in drinking water (EPA, 2011). Although the methods described above are highly sensitive and informative, they require specialized laboratory equipment, qualified personnel and have a high cost. Plate counting is a traditional yet very accurate method to detect total bacteria in liquid samples. The disadvantage of plate counting is that it requires specialized laboratory equipment and qualified personnel to perform the test. In addition, plate counting often requires a relatively long time to get results. None of these methods is at one and the same time accurate, rapid, usable in the field and cost effective. Therefore, there still exists a strong demand for a novel method for the detecting total bacteria in liquid samples having all the qualities defined previously.

### BRIEF SUMMARY OF THE INVENTION

The present invention comprises an in-vitro diagnostic device for the detection of viable bacteria in liquid sample within 24 hours. The vacuum ampoule viable bacteria detection system provides an all-in-one rapid detection test without any sophisticated laboratory equipment and further laboratory test. The method described herein can be performed in the field completely by personnel without specific microbiology training. The method and compositions described herein is based on the visible color change of liquid sample and capable of indicating CFU/mL (colony formation unit per milliliter) ranging from <10 to 10⁸ CFU/mL. The positivity of the present invention can also be measured with a UV spectrometer with will give a more accurate analysis of the bacteria concentration in the liquid sample.

### BRIEF DESCRIPTION OF THE DRAWINGS

FIG. 1-FIG. 1 depicts one example of the self-filling vacuum ampoule.

FIG. 2-FIG. 2 illustrates the negative vs positive results of the total viable bacteria ampoule test.

FIG. 3-FIG. 3 shows the reaction time vs bacterial density relationship under suggested conditions for the total viable bacteria ampoule test.

FIG. 4-FIG. 4 shows the visual results of the total bacteria ampoule test from low to high bacterial densities.

FIG. 5-FIG. 5 shows the absorbance spectrum of the total viable bacteria ampoule test with and without TTC.

FIG. 6-FIG. 6 shows the absorbance spectrum of the total viable bacteria ampoule test over time.

FIG. 7-FIG. 7 illustrates the negative vs positive results of the E. coli ampoule test

FIG. 8-FIG. 8 shows the reaction time vs bacterial density relationship under suggested conditions for the E. coli ampoule test.

FIG. 9-FIG. 9 shows the fluorescent spectrum of the E. coli ampoule test with and without 4MUG.

FIG. 10-FIG. 10 shows the fluorescent spectrum of the E. coli ampoule test overtime.

FIG. 11-FIG. 11 illustrates the general steps to conduct a bacteria test using the self-filling vacuum ampoule.

### DETAILED DESCRIPTION OF THE INVENTION

Unless otherwise defined, all terms (including technical and scientific terms) used herein have the same meaning as commonly understood by one having ordinary skill in the art to which this invention belongs. It will be further understood that terms, such as those defined commonly used in dictionaries, should be interpreted as having a meaning that is consistent with their meaning in the context of the relevant art and the present disclosure and will not be interpreted in an idealized or overly formal sense unless expressly so defined.

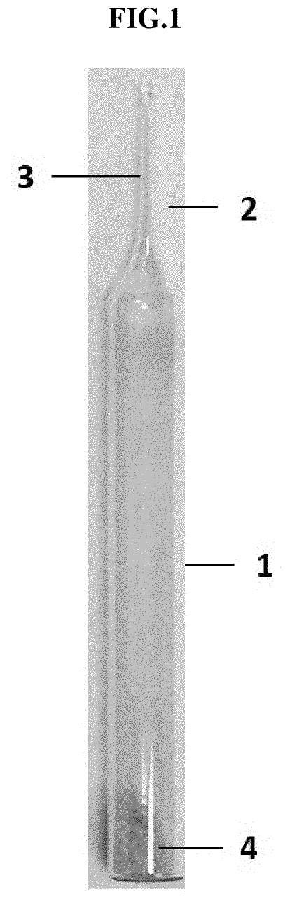

As shown in FIG. 1, the vacuum ampoule total bacteria detection system including a glass self-filling ampoule and the enclosed supporting medium and chemical indicator. The cylindrical glass ampoule body 1 has a length of 3.5 inches and a diameter of 0.45 inches. The ampoule neck 2 has a length of 1.5 inches. Etching point 3 is in the middle of the ampoule neck (0.75 inches from the tip of the ampoule neck). Supporting medium, selective reagent(s) and detection reagent(s) are enclosed in the vacuum ampoule.

The invention includes a vacuum ampoule which can take in about 7 mL liquid sample upon breaking the glass tip, a supporting medium that provides nutrients for bacterial culturing, selective reagent(s) that inhibit the growth of non-interested microbial species, and detection reagent(s) that indicates the presence of bacteria and provides indication of the cell density are included in the vacuum ampoule.

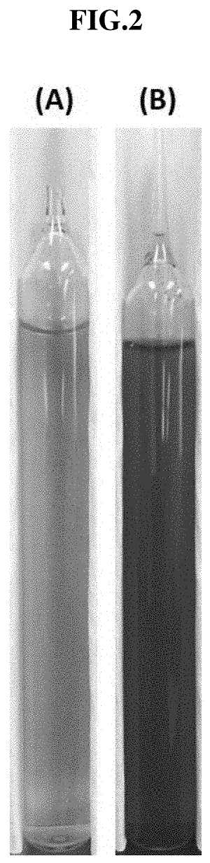

The total viable bacteria ampoule contains 1˜20% yeast extract, 10˜40% peptone, 10˜40% sodium chloride, 1˜10% lab-lemco powder, 0.1˜1% 2,3,5-triphenyltetrazolium chloride (TTC). In FIG. 2, (A) The negative control was filled with about 7 mL of sterile milli-Q water. (B) The glass ampoule was self-filled with about 7 mL 10⁸ CFU/mL mixed viable cell suspension of E. coli ATCC 25922 and Staphylococcus aureus (S. aureus) ATCC 25923. The ampoule was incubated at 37° C. for 1.5 hours. Digital photographs representing the real testing result without modification.

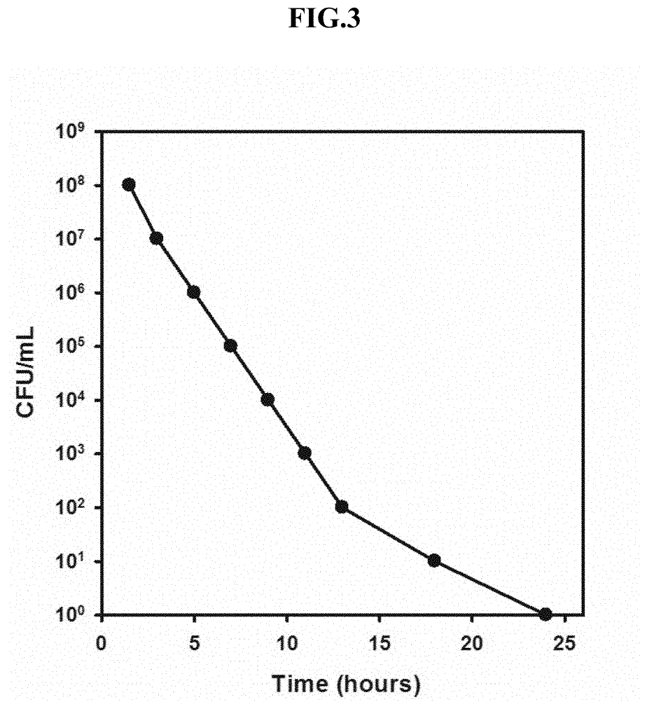

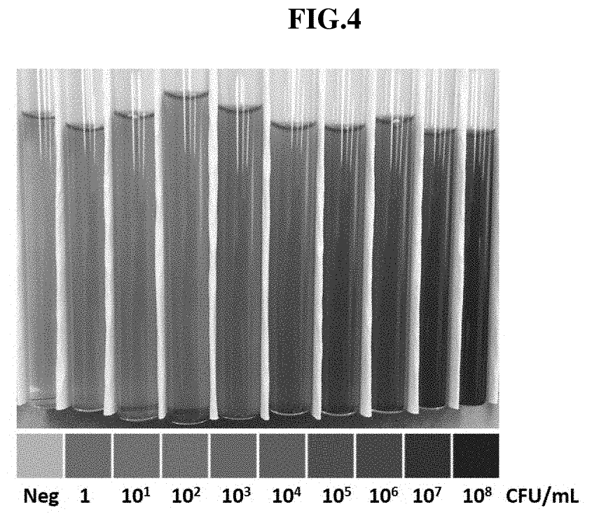

The semi-quantitative results of the total viable bacteria vacuum ampoule (FIG. 3) shows a negatively proportional correlation between the elapsed time of the appearance of the pink-red color and viable cell density (CFU/mL) of E. coli ATCC 25922 and S. aureus ATCC 25923 in the liquid sample. Ampoules were incubated 37° C. The elapsed time of the test ranges from 1.5 hours to 24 hours. Data variance is +/−1 hour. Visual results of the total viable bacteria ampoule test (FIG. 4) shows a positive proportional correlation between the intensity of the pink-red color and viable cell density (CFU/mL) of E. coli ATCC 25922 and S. aureus ATCC 25923 in the liquid sample. Ampoules were incubated at 37° C. for 24 hours. Digital photographs representing the real testing result without modification. Color gradient reference was generated based on real photographs.

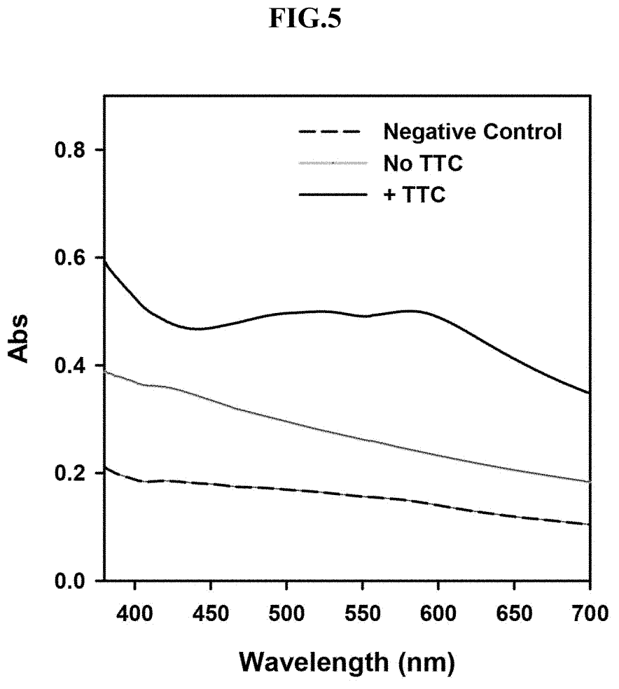

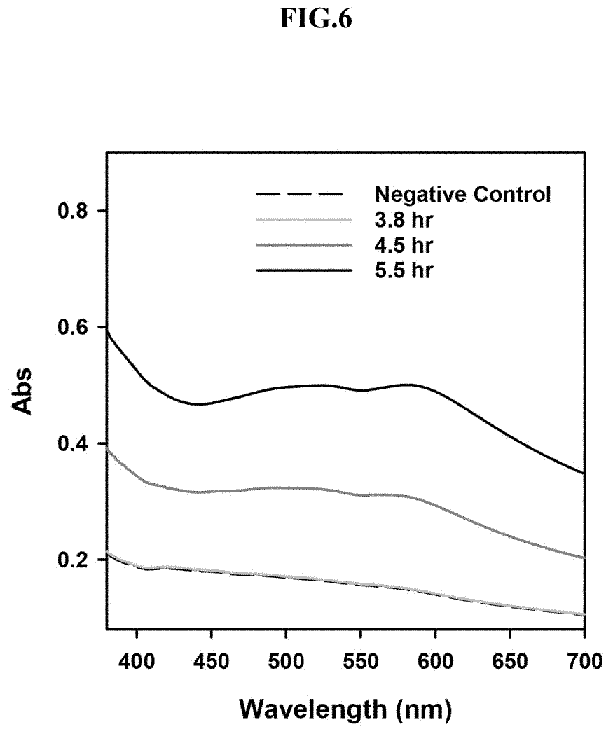

The total viable bacteria test result can be quantified using a UV spectrometer. As shown in FIG. 5, the total viable bacteria test was performed using 10⁶ CFU/mL as the initial inoculation concentration with the presence of TTC. In the negative control, no bacteria was inoculated. In the “No TTC” group, same bacterial concentration was applied without adding TTC. An absorbance peak was observed at 581 nm in the “+TTC” group but not in “No TTC” and “Negative Control” groups. The appearance of the pink-red color is an accumulative effect, i.e. as the reaction between bacteria and TTC continues, the color will become darker. This is confirmed by following the development of the absorbance spectrum development overtime (FIG. 6). With same initial bacterial inoculation, the absorbance peak at 581 nm grows as the incubation time increases.

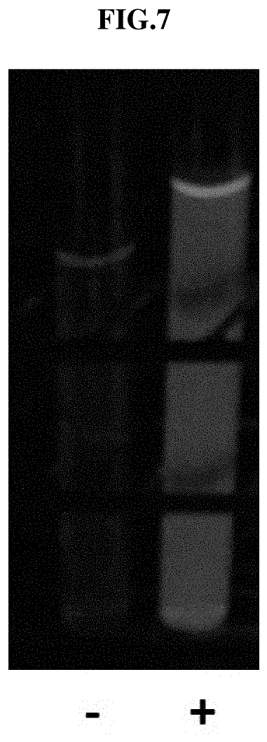

The E. coli ampoule contains 1˜20% yeast extract, 10˜40% peptone, 10˜40% sodium chloride, 1˜10% lab-lemco powder, 0.1˜1% 4-Methylumbelliferyl-β-D-glucuronide hydrate (4-MUG). In FIG. 7, the negative control (“−”) was filled with about 7 mL of sterile milli-Q water. The positive ampoule (“+”) self-filled with about 7 mL 10⁸ CFU/mL mixed viable cell suspension of E. coli ATCC 25922 showed strong blue fluorescence under the long-wave UV light. The ampoule was incubated at 37° C. for 24 hours. Digital photographs representing the real testing result without modification.

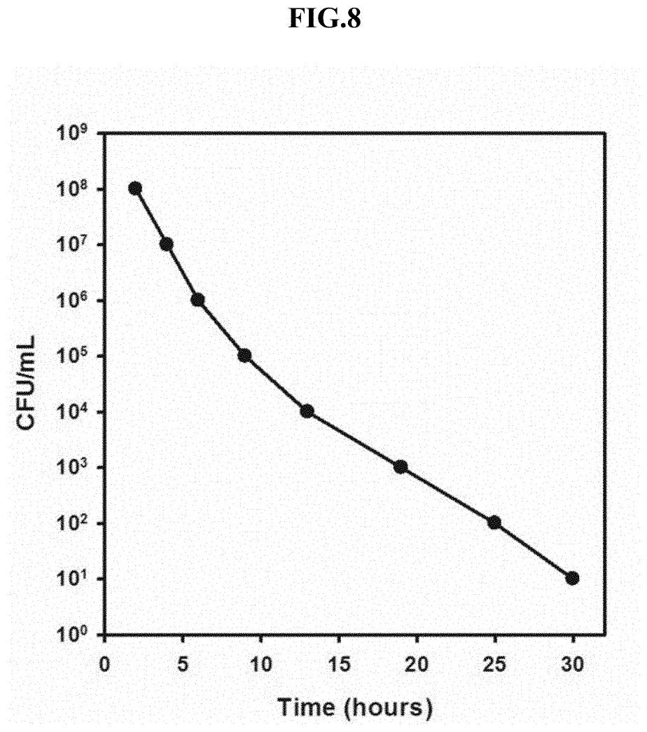

The semi-quantitative results of the E. coli ampoule test (FIG. 8) shows a negatively proportional correlation between the elapsed time of the appearance of the blue fluorescence and viable cell density (CFU/mL) of E. coli ATCC 25922 in the liquid sample. Ampoules were incubated 37° C. The elapsed time of the test ranges from 2 hours to 30 hours. Data variance is +/−1 hour.

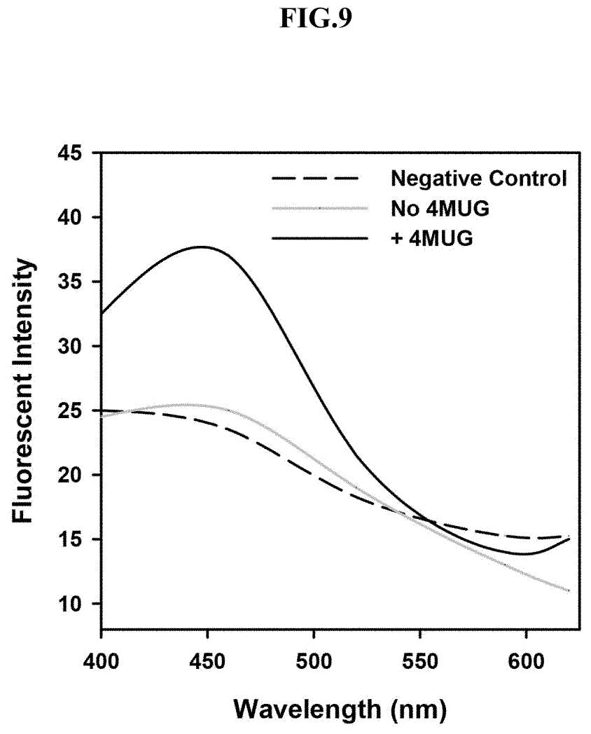

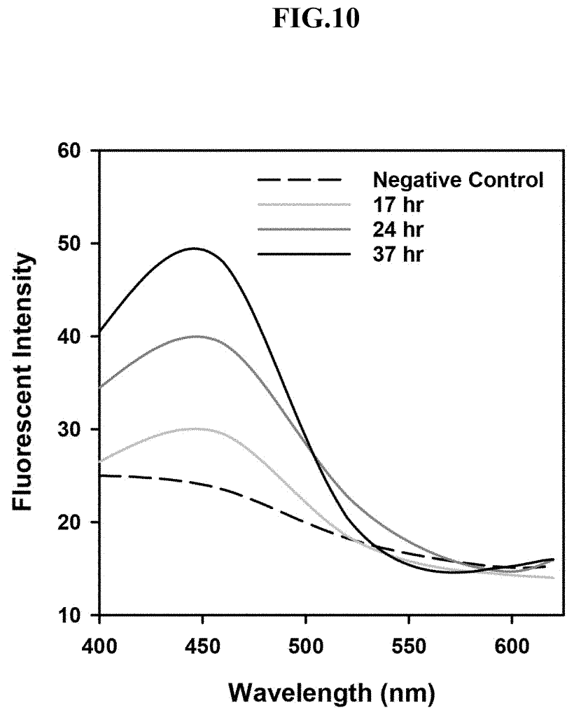

The formation of blue fluorescence in the E. coli ampoule test can also be captured and quantified using a fluorescent spectrometer. As shown in FIG. 9, the E. coli ampoule test was performed using 10⁶ CFU/mL as the initial inoculation concentration with the presence of 4MUG. In the negative control, no bacteria was inoculated. In the “No 4MUG” group, same bacterial concentration was applied without adding 4MUG. The emission spectrums of E. coli ampoule tests with and without 4MUG was captured using the 355 nm excitation. An emission peak at 460 nm was observed only with the presence of 4MUG. The formation of this emission peak at 460 nm is due to reaction between E. coli and 4MUG which forms a fluorescent product. The appearance of the blue fluorescence is an accumulative effect, i.e. as the reaction between bacteria and 4MUG continues, the fluorescence will become more intense. This is confirmed by following the development of the fluorescent spectrum development overtime (FIG. 10). With same initial bacterial inoculation, the emission peak at 460 nm grows as the incubation time increases.

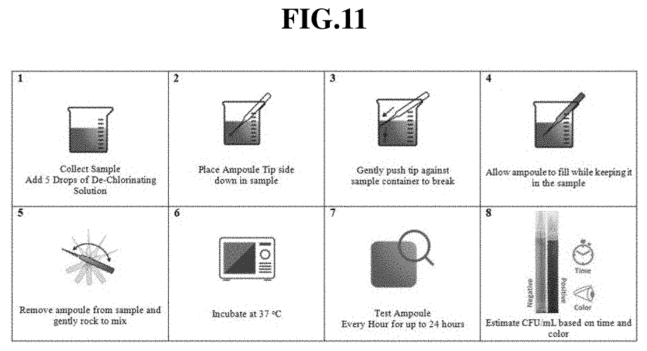

The general procedure (FIG. 11) of using the self-filling vacuum ampoule detection system to determine the specific bacteria type in liquid sample includes: 1. Collect sample in a container. Add de-chlorination liquid if chlorine is present in the sample. 2. Place the ampoule tip side down in the sample. 3. Gently push tip against sample container to break. 4. Allow ampoule to fill while keeping it in the sample. 5. Remove ampoule from sample container and gently rock and mix. 6. Incubate at 37° C. 7. Test ampoule hourly for up to 30 hours. 8. Estimate CFU/mL based on elapsed time and color intensity.

1. A self-filling vacuum ampoule detection system to rapidly quantify viable total heterotrophic bacteria in a liquid sample, the self-filling vacuum ampoule detection system comprising:
a supporting medium, wherein the supporting medium comprises nutrients for culture bacterial species in the liquid samples;
at least one selective reagent to inhibit a growth of interference microbial species in the liquid sample; and
a detection reagent to quantify an amount of specific bacterial species in the liquid sample.

2. A self-filling vacuum ampoule detection system to rapidly quantify viable total heterotrophic bacteria in a liquid sample, the self-filling vacuum ampoule detection system comprising:
the self-filling vacuum ampoule that comprises:
a supporting medium to provide nutrients for the viable total heterotrophic bacteria in the liquid sample, and
a detection reagent to quantify a density of the viable total heterotrophic bacteria in the liquid sample,
wherein the self-filling vacuum ampoule contains about 7 mL of the liquid sample.

3. The self-filling vacuum ampoule detection system of claim 2,
wherein the self-filling vacuum ampoule comprises 1˜20% of yeast extract, 10˜40% of peptone, 10˜40% of sodium chloride, 1˜10% of lab-lemco powder, and 0.1˜1% of 2,3,5-triphenyltetrazolium chloride (TTC) and detects the viable total heterotrophic bacteria in the liquid samples.

4. The self-filling vacuum ampoule detection system of claim 2,
wherein the self-filling vacuum ampoule comprises 1˜20% of yeast extract, 10˜40% of peptone, 10˜40% of sodium chloride, 1˜10% of lab-lemco powder, and 0.1˜1% of 4-Methylumbelliferyl-β-D-glucuronide hydrate (4-MUG), and detects E. coli bacteria in the liquid samples.

5. A method to identify the positivity of the self-filling vacuum ampoule detection system of claim 2, the method comprising:
obtaining an absorbance spectra measurement of the self-filling vacuum ampoule; and
identifying an absorbance peak at 581 nm wavelength.

6. A method to identify the positivity of the self-filling vacuum ampoule detection system of claim 2, the self-filling vacuum ampoule detection system detecting E. coli bacteria in the liquid sample, the method comprising:
obtaining a fluorescence measurement of the self-filling vacuum ampoule with light in 355 nm excitation wavelength; and
identifying an emission peak at 460 nm wavelength.
